(19) United States
(12) Patent Application Publication
(10) Pub. No.: US 2011/0098816 A1
(43) Pub. Date: Apr. 28, 2011

(54) SACRO-ILIAC JOINT IMPLANT SYSTEM AND METHOD

(75) Inventors: Clinton R. Jacob, Germantown, TN (US); Jason J. Eckhardt, Memphis, TN (US); Mark Dace, Collierville, TN (US)

(73) Assignee: WARSAW ORTHOPEDIC, INC., Warsaw, IN (US)

(21) Appl. No.: 12/607,453

(22) Filed: Oct. 28, 2009

(51) Int. Cl. A61F 2/44

(52) U.S. Cl. 623/17.11

(57) ABSTRACT

An orthopedic implant includes at least one circular body. The at least one circular body defining an outer surface configured to engage at least one articular surface of a sacro-iliac joint along a plane substantially parallel to the articular surface.

### TECHNICAL FIELD

The present disclosure generally relates to medical devices for the treatment of musculoskeletal disorders, and more particularly to an implant system and method for treating the sacro-iliac joint.

### BACKGROUND

The sacroiliac (SI) joint is a diarthrodial joint that joins the sacrum to the ilium bones of the pelvis. In the SI joint, the sacral surface has hyaline cartilage that moves against fibrocartilage of the iliac surface. The spinal column is configured so that the weight of an upper body rests on the SI joints at the juncture of the sacrum and ilia. Stress placed on the SI joints in an upright position of the body makes the lower back susceptible to injury.

Disorders of the SI joint can cause low back and radiating buttock and leg pain in patients suffering from degeneration and laxity of the SI joint. In some cases, the SI joint can undergo dehydration and destabilization, similar to other cartilaginous joints, which causes significant pain. The SI joint is also susceptible to trauma and degeneration, from fracture and instability. It is estimated that disorders of the SI joint are a source of pain for millions of people suffering from back and radicular symptoms.

Non-surgical treatments, such as medication, injection, mobilization, rehabilitation and exercise can be effective, however, may fail to relieve the symptoms associated with these disorders. Surgical treatment of these disorders include stabilization and/or arthrodesis. Stabilization can include the use of bone screws that are directly threaded into bone. Arthrodesis may include fusion devices to immobilize a joint. The present disclosure describes an improvement over these prior art technologies.

### SUMMARY OF THE INVENTION

Accordingly, an implant system and method is provided for treating the SI joint. It is contemplated that the system may include an implant configured as a SI joint spacer. It is further contemplated that the implant system and method may be employed for arthrodesis and/or arthroplasty treatment.

In one particular embodiment, in accordance with the principles of the present disclosure, an orthopedic implant is provided. The orthopedic implant includes at least one circular body. The at least one circular body defining an outer surface configured to engage at least one articular surface of a sacro-iliac joint along a plane substantially parallel to the articular surface.

### BRIEF DESCRIPTION OF THE DRAWINGS

The present disclosure will become more readily apparent from the specific description accompanied by the following drawings, in which:

FIG. 1 is a perspective view of one particular embodiment of an implant system in accordance with the principles of the present disclosure;

FIG. 1A is a perspective view of one embodiment of the implant system shown in FIG. 1;

FIG. 2 is a plan view of the implant system shown in FIG. 1 and a SI joint;

FIG. 3 is an enlarged perspective view of one embodiment of an implant system in accordance with the principles of the present disclosure;

FIG. 4 is a perspective view of the implant system shown in FIG. 3;

FIG. 5 is a side, cross-section view of the implant system shown in FIG. 3;

FIG. 6 is an enlarged perspective view of one embodiment of an implant system;

FIG. 7 is a plan view of the implant system shown in FIG. 3 and a SI joint;

FIG. 8 is a plan view of a SI joint illustrating preparation device orientation;

FIG. 9 is a plan view of one embodiment of an implant system and a SI joint;

FIG. 10 is a plan view of a SI joint illustrating one embodiment of SI joint cavity preparation;

FIG. 11 is a plan view of a SI joint illustrating one embodiment of SI joint cavity preparation;

FIG. 12 is a plan view of a SI joint illustrating one embodiment of SI joint cavity preparation;

FIG. 13 is a plan view of a SI joint illustrating one embodiment of SI joint cavity preparation;

FIG. 14 is a perspective view of one embodiment of an implant system in accordance with the principles of the present disclosure;

FIG. 15 is a perspective view of the implant system shown in FIG. 14 with an implantation device;

FIG. 16 is a perspective view of a portion of one embodiment of the implant system shown in FIG. 14;

FIG. 17 is a perspective view of one embodiment of an implant system in accordance with the principles of the present disclosure; and

FIG. 18 is a perspective view of the implant system shown in FIG. 17 with an implantation device.

Like reference numerals indicate similar parts throughout the figures.

### DETAILED DESCRIPTION OF THE INVENTION

The exemplary embodiments of the implant system and methods of use disclosed are discussed in terms of medical devices for the treatment of musculoskeletal disorders and more particularly, in terms of an implant system and method for treating the SI joint. It is envisioned that the implant system and methods of use disclosed provide stability and maintains structural integrity while reducing stress on the SI joint. It is further envisioned that the present disclosure may be employed to treat musculoskeletal disorders including sacro-Iliac dysfunction or syndrome, dehydration, destabilization, laxity, fracture, tumor, spinal disorders and other orthopedic disorders. It is contemplated that the present disclosure may be employed with surgical treatments, including open surgery, percutaneous and minimally invasive procedures of such disorders, such as, for example, arthrodesis including fusion, arthroplasty to maintain motion, bone graft and implantable prosthetics. It is further contemplated that the present disclosure may be employed with other osteal and bone related applications, including those associated with diagnostics and therapeutics. The disclosed implant system and methods may be employed in a surgical treatment with a patient in a prone or supine position, employing a posterior, lateral, inferior, posterior-inferior, superior or anterior approach. The present disclosure may be employed with procedures for treating the lumbar, cervical, thoracic and pelvic regions of a spinal column.

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

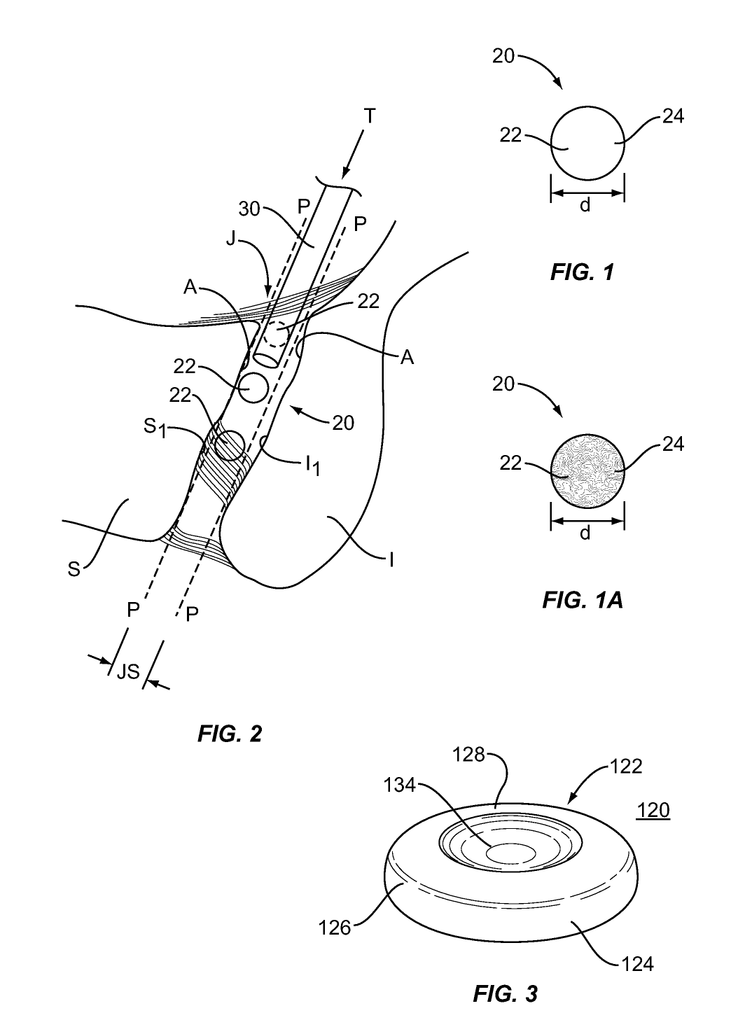

The following discussion includes a description of an implant system, related components and exemplary methods of employing the implant system in accordance with the principles of the present disclosure. Alternate embodiments are also disclosed. Reference will now be made in detail to the exemplary embodiments of the present disclosure, which are illustrated in the accompanying figures. Turning now to FIGS. 1 and 2, there are illustrated components of the implant system in accordance with the principles of the present disclosure.

The components of the implant system are fabricated from materials suitable for medical applications, including metals, synthetic polymers, ceramics, bone, biocompatible materials and/or their composites, depending on the particular application and/or preference of a medical practitioner. For example, components of the implant system, such as, for example, a circular body, an outer surface of the circular body and/or portions thereof, discussed below, can be fabricated from materials such as commercially pure titanium, titanium alloys, super-elastic titanium alloys, cobalt-chrome alloys, stainless steel alloys, thermoplastics such as polyaryletherketone (PAEK) including polyetheretherketone (PEEK), polyetherketoneketone (PEKK) and polyetherketone (PEK), carbon fiber reinforced PEEK composites, PEEK-BaSO₄ composites, ceramics and composites thereof, rigid polymers including polyphenylene, polyamide, polyimide, polyetherimide, polyethylene, polyurethanes of any durometer, epoxy, silicone, bone material including autograft, allograft, xenograft or transgenic cortical and/or corticocancellous bone, and tissue growth or differentiation factors. Different components of the implant system may have alternative material composites to achieve various desired characteristics such as strength, rigidity, elasticity, compliance, biomechanical performance, durability and radiolucency or imaging preference.

It is envisioned that the components of the implant system can be manufactured via various methods. For example, the circular body can be manufactured and assembled via injection-molding, insert-molding, overmolding, compression molding, transfer molding, co-extrusion, pultrusion, dip-coating, spray-coating, powder-coating, porous-coating, milling from a solid stock material, and their combinations. One skilled in the art, however, will realize that such materials and fabrication methods suitable for assembly and manufacture, in accordance with the present disclosure, would be appropriate.

The implant system includes an orthopedic implant, such as, for example, a sacro-iliac implant 20, which is configured, for example, to treat S-I joint disorders including those caused by degeneration or trauma. It is contemplated that sacro-iliac implant 20 may be employed for arthrodesis and/or arthroplasty applications, as will be described.

Sacro-iliac implant 20 includes a circular body, such as, for example, spherical body 22 that defines an outer surface 24. Outer surface 24 is configured to engage an articular surface A of a sacro-iliac joint J along a plane P. Plane P is substantially parallel to articular surface A. It is contemplated that articular surface A may refer to a sacral surface S₁ of a sacrum S and/or an iliac surface I₁ of an ilium I. Body 22 may be configured to engage opposing articular surfaces such as sacral surface S₁ and iliac surface I₁ and/or opposing valleys or peaks of an individual sacrum S or ilium I. Body 22 may have a ball, solid, hollow, porous or cage configuration. Outer surface 24 has a continuously even or smooth configuration. It is contemplated that outer surface 24 is configured to substantially match articular surface(s) A and may be substantially smooth, rough and/or polished. In one embodiment, as shown in FIG. 1A, an outer surface has a textured configuration.

Body 22 has a circular cross section and has a diameter d, according to the requirements of the particular application. It is envisioned that diameter d may be in a range of 0.5-5.0 millimeters (mm). It is contemplated that the cross-sectional geometry of body 22 may have various configurations, for example, round, oval, rectangular, polygonal, irregular, uniform, non-uniform, consistent or variable.

It is envisioned that body 22 can be variously configured and dimensioned with regard to size, shape, thickness, geometry and material. Body 22 may also be formed of one or a plurality of elements such as spaced apart portions, staggered patterns and mesh. It is envisioned that the particular geometry and material parameters of body 22 may be selected to modulate the flexibility or stiffness of sacro-iliac implant 20, such as those examples discussed herein. For example, body 22 can be configured to have varying ranges or degrees of flexibility or stiffness such as rigid, compliant, or reinforced. Depending on the flexibility or stiffness of body 22, the flexibility or stiffness of sacro-iliac implant 20 can be contoured according to the requirements of a particular application. It is contemplated that the ability to vary stiffness of sacro-iliac implant 20 provides restoration of kinematic function of joint J or promote fusion of the elements of joint J. It is envisioned that the components of sacro-iliac implant 20 may be monolithically formed, integrally connected or arranged with attaching elements.

In one embodiment, as shown in FIG. 2, the implant system includes a plurality of spherical bodies 22, described above. It is contemplated that employing the plurality of bodies 22 can optimize the amount joint J can be spaced apart such that a joint space JS can be preselected. The plurality of bodies 22 can be inserted through the same or an alternate trajectory. The plurality of bodies 22 can be oriented in a side by side engagement, spaced apart and/or staggered. It is envisioned that one or all of the plurality of bodies 22 may be inserted via a trajectory oriented from an anterior, posterior, superior or inferior direction, similar to that described below with regard to FIG. 8. It is further envisioned that one or a plurality of bodies 22 may be used.

In assembly, operation and use, the implant system including sacro-iliac implant 20 is employed with a surgical procedure for treatment of a sacro-iliac joint of a patient, as discussed herein. The implant system may also be employed with other surgical procedures. In particular, the implant system is employed with a surgical arthroplasty procedure for treatment of an applicable condition or injury of an affected sacro-iliac joint J, as shown in FIG. 2. It is contemplated that the implant system is inserted with a sacro-iliac joint to provide a less invasive approach for treatment and re-establish joint tension. It is further contemplated that the implant system is inserted with a sacro-iliac joint as a SI joint spacer to restore ligamentous tension, eliminate painful micro-motion and/or preserve motion. It is envisioned that the implant system may maintain joint tension without promoting bone growth.

In use, to treat the affected section of sacro-iliac joint J, a medical practitioner obtains access to a surgical site including sacro-iliac joint J in any appropriate manner, such as through incision and retraction of tissues. It is envisioned that the implant system may be used in any existing surgical method or technique including open surgery, mini-open surgery, minimally invasive surgery and percutaneous surgical implantation, whereby sacro-iliac joint J is accessed through a mini-incision, or sleeve that provides a protected passageway to the area. Once access to the surgical site is obtained, the particular surgical procedure is performed for treating the sacro-iliac joint disorder. The implant system is then employed to augment the surgical treatment. The implant system can be delivered or implanted as a pre-assembled device or can be assembled in situ. The implant system may be completely or partially revised, removed or replaced in situ.

A trajectory T is defined for insertion and/or injection of sacro-iliac implant 20 within sacro-iliac joint J. Trajectory T is determined by defining an orientation that is substantially parallel to plane P defined by articular surface A of sacro-iliac joint J. Implant 20 is inserted via the protected passageway along the defined trajectory T into sacro-iliac joint J. A cavity of sacro-iliac joint J is prepared along trajectory T for disposal of sacro-iliac implant 20.

The protected passageway includes a dilator/delivery tube 30 configured to deliver sacro-iliac implant 20 directly to joint space JS of sacro-iliac joint J. It is envisioned that dilator/delivery tube 30 may be configured as an in-situ guidable instrument, and may include an endoscope camera tip for viewing insertion trajectory.

Sacro-iliac implant 20 is manipulated such that outer surface 24 of body 22 engages articular surface A substantially along plane P, according to the contour of articular surface A. Manipulation can include pushing, pulling, rotation of sacro-iliac implant 20, rotation of sacro-iliac implant 20 about the joint axis once implanted and/or by mechanical devices.

Sacro-iliac implant 20 is disposed with sacro-iliac joint J for treating the sacro-iliac joint disorder. Body 22 is configured for movable engagement with articular surfaces A in a plurality of directions. Bodies 22 roll along articular surface A allowing relative motion of the articular surfaces A of the sacrum and ilium of sacro-iliac joint J. The outer surfaces of bodies 22 may be compressible. It is envisioned that body 22 may be inserted via a trajectory oriented from a posterior, lateral, inferior, posterior-inferior, superior or anterior direction.

It is contemplated that the implant system including sacro-iliac implant 20 may be employed during a surgical fusion procedure for treatment of a condition or injury, such as, degeneration or fracture. Fixation of sacro-iliac implant 20 with articular surface A and/or other portions of sacro-iliac joint J can be facilitated by the resistance provided by joint space JS and/or engagement with the outer articular structures. Sacro-iliac implant 20 may include locking structure to facilitate fixation with articular surface(s) A. It is envisioned that such locking structure may include fastening elements such as, for example, clips, hooks, adhesives and/or flanges, as will be described below. It is further envisioned that in joint fusion applications of sacro-iliac implant 20, body 22 includes voids, cavities and/or openings for including bone growth promoting material, such as those described herein, which can be packed or otherwise disposed therein.

It is envisioned that implant system can be used with various bone screws to enhance fixation. It is contemplated that the implant system and any screws and attachments may be coated with an osteoconductive material such as hydroxyapatite and/or osteoinductive agent such as a bone morphogenic protein for enhanced bony fixation to facilitate motion of the treated area. Sacro-iliac implant 20 can be made of radiolucent materials such as polymers. Radiomarkers may be included for identification under x-ray, fluoroscopy, CT or other imaging techniques.

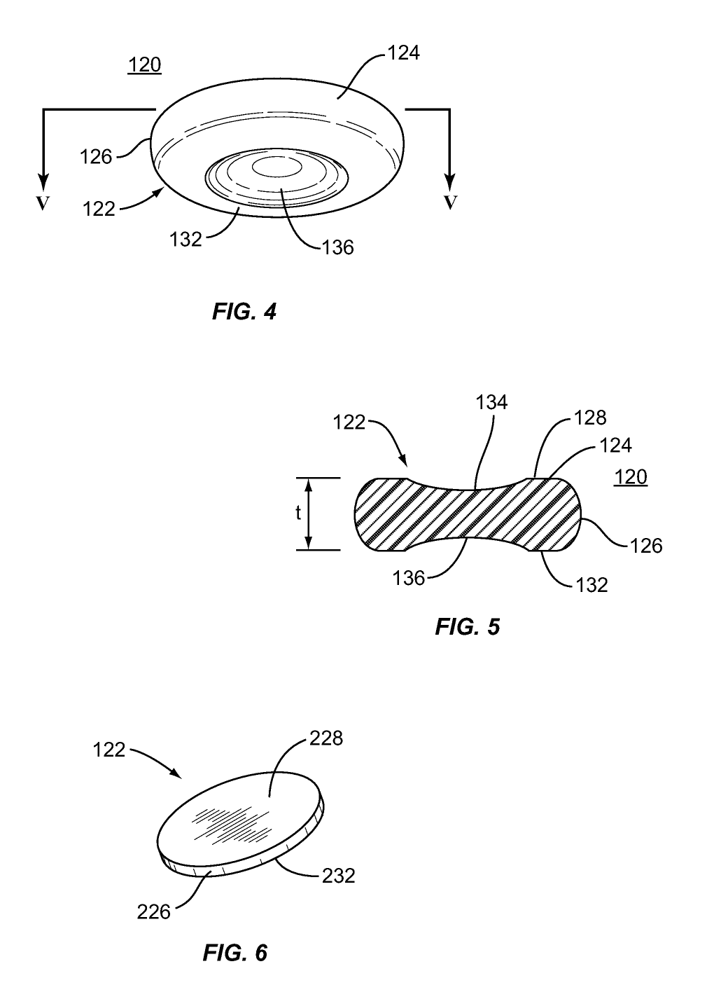

Referring to FIGS. 3-5, in one embodiment similar to the implant system described above, a sacro-iliac implant 120 is configured, for example, to treat S-I joint disorders. It is contemplated that sacro-iliac implant 120 may be employed for arthrodesis and/or arthroplasty applications, as will be described.

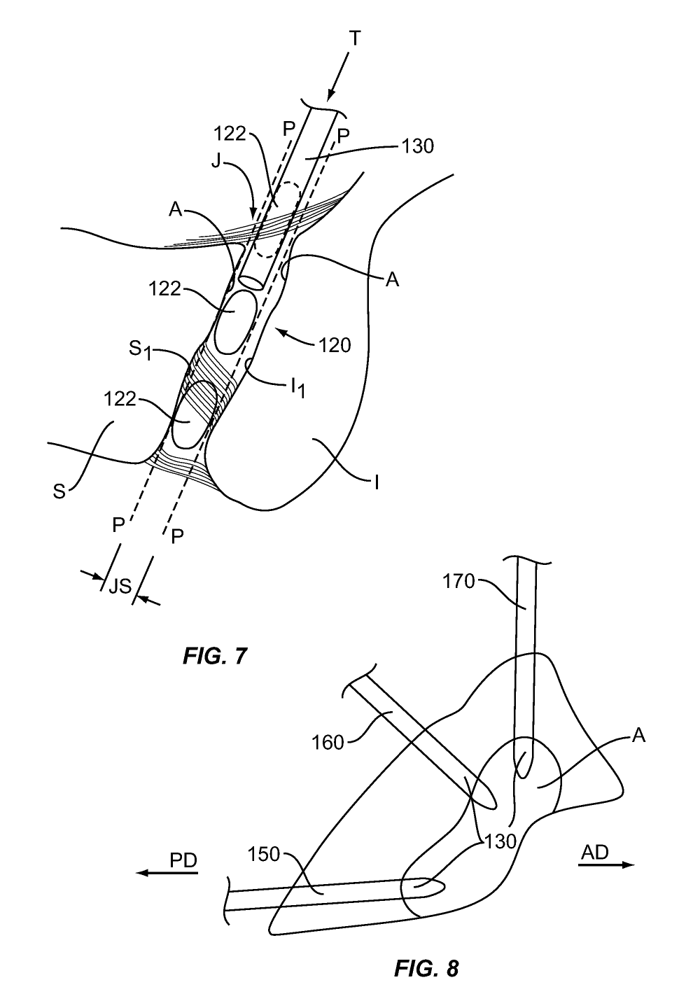

Sacro-iliac implant 120 includes a circular body, such as, for example, a disc 122, similar to body 22 discussed above, which defines an outer surface 124. Outer surface 124 is configured to engage an articular surface A of a sacro-iliac joint J along a plane P (FIG. 7). Plane P is substantially parallel to articular surface A. It is contemplated that articular surface A may refer to a sacral surface S1 of a sacrum S and/or an iliac surface I1 of an ilium I. Disc 122 may be configured to engage opposing articular surfaces such as sacral surface S₁ and iliac surface I₁ and/or opposing valleys or peaks of an individual sacrum S or ilium I. Disc 122 may have a solid, hollow or porous configuration.

Disc 122 has a periphery 126 that defines a thickness t of disc 122. It is envisioned that thickness t may be in a range of 0.5-5.0 mm. Periphery 126 has an arcuate configuration and is disposed about the perimeter of disc 122. It is envisioned that periphery 126 may have alternate cross section configurations such as elliptical, rectangular and polygonal. It is further envisioned that periphery 126 may define a substantially planar surface about the perimeter of disc 122.

Outer surface 124 includes a first lateral surface 128 and a second lateral surface 132. First lateral surface 128 opposes second lateral surface 132 about periphery 126. First lateral surface 128 defines a concave portion 134 and second lateral surface 132 defines an opposing concave portion 136. It is contemplated that only one of lateral surfaces 128, 132 may include a concave portion and/or may include alternate surface configurations. Lateral surfaces 128, 132 may each include one or a plurality of spaced apart concave portions. It is further contemplated that periphery 126 and/or lateral surfaces 128, 132 may include a concave, convex, planar, textured and/or perforated configuration. In one embodiment, as shown in FIG. 6, disc 122 includes a planar periphery 226, and a first lateral surface 228 and a second lateral surface 232, each of which having a substantially planar configuration. Disc 122 can be delivered to a sacro-iliac joint J via a dilator/delivery tube having an oval cross section to increase a distance between articulating surfaces A of sacro-iliac joint J. Disc 122 may also be delivered via a grasp inserter that engages sacro-iliac implant 120.

Depending on the flexibility or stiffness of disc 122, the flexibility or stiffness of sacro-iliac implant 120 can be configured according to the requirements of a particular application. It is contemplated that the ability to vary stiffness of sacro-iliac implant 120 provides restoration of kinematic function of sacro-iliac joint J or promotes fusion of the elements of sacro-iliac joint J.

In one embodiment, as shown in FIG. 7, the implant system includes a plurality of discs 122, described above. It is contemplated that employing the plurality of discs 122 can optimize the amount joint J can be spaced apart such that joint space JS can be preselected. The plurality of discs 122 can be inserted through the same or an alternate trajectory. The plurality of discs 122 can be oriented in a side by side engagement, spaced apart and/or staggered. It is envisioned that one or all of the plurality of discs 122 may be inserted via a trajectory oriented from an anterior, posterior, superior or inferior directions, similar to that described below with regard to FIG. 8. It is further envisioned that one or a plurality of discs 122 may be used.

In assembly, operation and use, the implant system including sacro-iliac implant 120 is employed with a surgical procedure for treatment of a sacro-iliac joint of a patient and may be employed with other surgical procedures. Referring to FIG. 7, the implant system is employed with a surgical fusion procedure for treatment of a condition or injury of sacro-iliac joint J, similar to that described with regard to FIG. 2. In use, to treat the affected section of sacro-iliac joint J, a medical practitioner obtains access to a surgical site including sacro-iliac joint J.

A trajectory T is defined for insertion of sacro-iliac implant 120 within sacro-iliac joint J. Trajectory T is determined by defining an orientation that is substantially parallel to plane P defined by articular surface A of sacro-iliac joint J. In other embodiments, as shown in FIG. 8 with arrow PD illustrating a posterior direction and arrow AD illustrating an anterior direction, alternate trajectory approach angles are shown. For example, the joint angle approach for sacro-iliac joint J preparation and delivery of a dilator/delivery tube 130 can include a posterior approach 150, a posterior-inferior approach 160 and/or an inferior approach 170.

Implant 120 is inserted with dilator/delivery tube 130 via a protected passageway along the defined trajectory T into sacro-iliac joint J. A cavity of sacro-iliac joint J is prepared along trajectory T for disposal of sacro-iliac implant 20. Disc 122 may also be delivered via a grasp inserter that engages sacro-iliac implant 120.

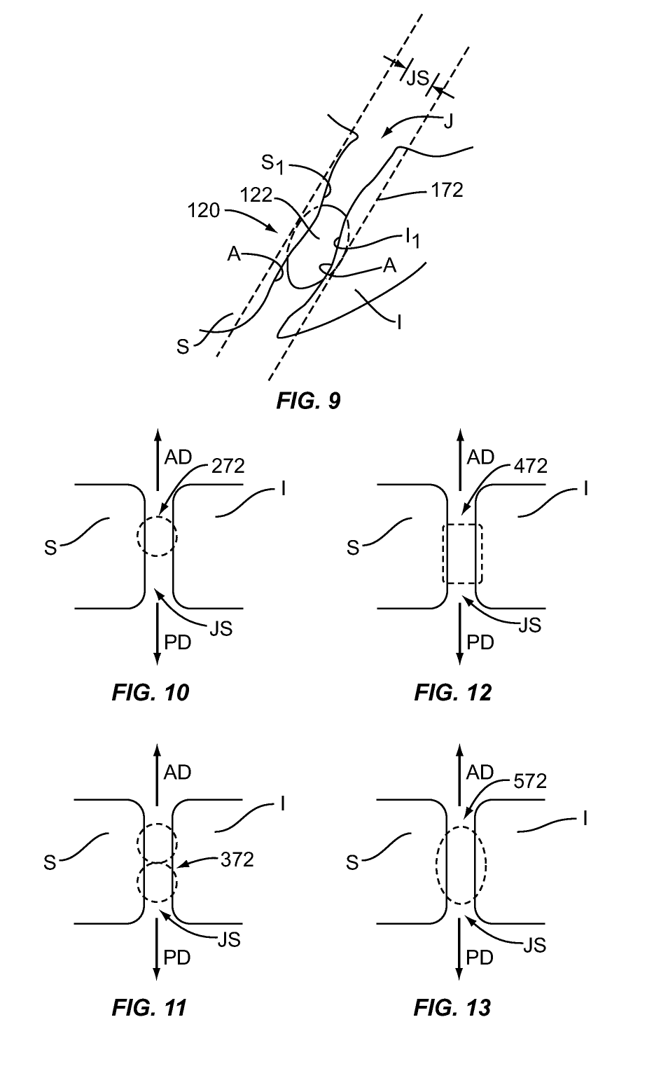

In one embodiment, as shown in FIG. 9, an oversized cavity 172 is created in joint space JS via a surgical preparation for disposal of sacro-iliac implant 120, implant devices and/or components of the protected passageway. Cavity 172 can be created via drilling, rasping, reaming, cutting, boring and/or surgical cutting methods for removing bone and other tissue. Sacro-iliac implant 120 has a compressible configuration and is oversized relative to cavity 172 to provide joint spacing and ligament tension to sacro-iliac joint J.

In one embodiment, as shown in FIG. 10 with arrow PD illustrating a posterior direction and arrow AD illustrating an anterior direction, a cavity 272 is created in joint space JS via a surgical preparation for disposal of sacro-iliac implant 120, implant devices and/or components of the protected passageway. Cavity 272 has a circular cross section and is created via a drilling device.

In one embodiment, as shown in FIG. 11 with arrow PD illustrating a posterior direction and arrow AD illustrating an anterior direction, multiple cavities 372 are created in joint space JS via a surgical preparation for disposal of sacro-iliac implant 120, implant devices and/or components of the protected passageway. Cavities 372 each have a circular cross section and are created via a drilling device.

In one embodiment, as shown in FIG. 12 with arrow PD illustrating a posterior direction and arrow AD illustrating an anterior direction, a cavity 472 is created in joint space JS via a surgical preparation for disposal of sacro-iliac implant 120, implant devices and/or components of the protected passageway. Cavity 472 has a rectangular cross section and is created via a rectangular or square rasping device.

In one embodiment, as shown in FIG. 13 with arrow PD illustrating a posterior direction and arrow AD illustrating an anterior direction, a cavity 572 is created in joint space JS via a surgical preparation for disposal of sacro-iliac implant 120, implant devices and/or components of the protected passageway. Cavity 572 has an elliptical cross section and is created via a an elliptical rasping device.

Referring to FIG. 7, the protected passageway includes a dilator/delivery tube 130 configured to deliver sacro-iliac implant 120 directly to joint space JS of sacro-iliac joint J. It is envisioned that dilator/delivery tube 130 may be configured as an in-situ guidable instrument, and may include an endoscope camera tip for viewing insertion trajectory.

Sacro-iliac implant 120 is manipulated such that outer surface 124 of body 122 engages articular surface A along plane P. Sacro-iliac implant 120 is disposed with sacro-iliac joint J for treating the sacro-iliac joint disorder. Sacro-iliac implant 120 may include locking structure to facilitate fixation with articular surface(s) A. It is envisioned that in joint fusion applications of sacro-iliac implant 120, disc 122 includes voids, cavities and/or openings for including bone promoting material, such as those described herein, which can be packed or otherwise disposed therein.

It is contemplated that the implant system including sacro-iliac implant 120 may be employed during arthoplasty. For example, disc 122 is configured for movable engagement with articular surfaces A. Discs 122 facilitate relative movement of articular surface(s) A allowing relative motion of the articular surfaces A of the sacrum and ilium of sacro-iliac joint J. The lateral surfaces of discs 122 may be compressible.

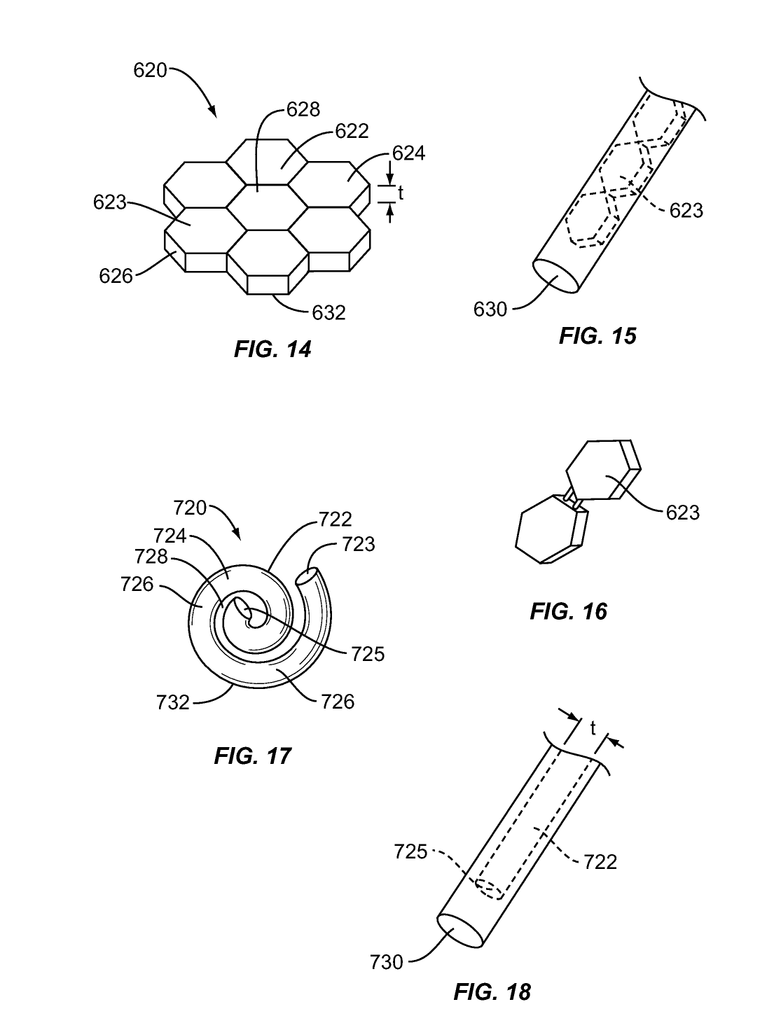

Referring to FIGS. 14 and 15, in one embodiment similar to the implant system described above, a sacro-iliac implant 620 is configured, for example, to treat S-I joint disorders. It is contemplated that sacro-iliac implant 620 may be employed for arthrodesis and/or arthroplasty applications, as will be described.

Sacro-iliac implant 620 includes a circular body, such as, for example, a segmented disc 622, similar to disc 122 discussed above, which defines an outer surface 624. Outer surface 624 is configured to engage articular surface(s) A of sacro-iliac joint J along plane(s) P (FIG. 7). Disc 622 has a periphery 626 that defines a thickness t of disc 622. It is envisioned that thickness t may be in a range of 0.5-5.0 mm. Periphery 126 has substantially planar surface and is disposed about the perimeter of disc 622.

Outer surface 624 includes a first lateral surface 628 and a second lateral surface 632. First lateral surface 628 and second lateral surface 632, each have a substantially planar configuration. First lateral surface 628 opposes second lateral surface 632 about periphery 626. It is contemplated that periphery 626 and/or lateral surfaces 628, 632 may include a concave, convex, planar, textured and/or perforated configuration.

Disc 622 is an elastic body that includes a plurality of segments 623. The segments of disc 622 are connected and relatively movable, such that the circular shape of disc 622 is deformable. The segments of disc 622 are manipulated and deformed from the circular disc shape into a substantially straightened, non-relaxed configuration, as shown in FIG. 15. This configuration of the segments of disc 622 allows for insertion and loading of the segments with a dilator/delivery tube 630 and subsequent delivery to sacro-iliac joint J, similar to that described. Disposal of the straightened segments of disc 622 with the dilator/delivery tube 630 maintains a deformed, linear orientation of disc 622. It is contemplated that segments 623 may include similar and alternatively configured segments. It is further contemplated that a plurality of discs 622 may be employed.

In assembly, operation and use, the implant system including sacro-iliac implant 620 is employed with a surgical procedure for treatment of a sacro-iliac joint of a patient and may be employed with other surgical procedures. It is contemplated that sacro-iliac implant 620 may be employed for arthrodesis and/or arthroplasty applications.

Dilator/delivery tube 630 is delivered with sacro-iliac implant 620 and the linear orientation of disc 622 disposed therein, via a protected passageway to joint space JS of sacro-iliac joint J. Disc 622 is unloaded from dilator/delivery tube 630 into joint space JS of sacro-iliac joint J. It is envisioned that disc 622 may have a segmented coil configuration that can be loaded into a large gauge needle and delivered to joint space JS. The protected passageway may include dilators.

As sacro-iliac implant 620 enters joint space JS, the segments of disc 622 are no longer subject to the restraint of tube 630 and deformation. The segments return to the unstressed, relaxed configuration such that folded segments 623 retain their disc shape, as shown in FIG. 14, due to the compliant configuration of disc 622. Sacro-iliac implant 620 is disposed with sacro-iliac joint J for treating the sacro-iliac joint disorder, similar to that described above. In one embodiment, as shown in FIG. 16, disc 622 includes segments 623 formed of a rigid material, such as those described herein, and are connected by elastics or springs, which may be fabricated from Nitinol or other shape memory material.

Referring to FIGS. 17 and 18, in one embodiment similar to the implant system described above, a sacro-iliac implant 720 is configured, for example, to treat S-I joint disorders. It is contemplated that sacro-iliac implant 720 may be employed for arthrodesis and/or arthroplasty applications, as will be described.

Sacro-iliac implant 720 includes a circular body, such as, for example, a coiled disc 722, similar to disc 122 discussed above, which defines an outer surface 724. Outer surface 724 is configured to engage articular surface(s) A of sacro-iliac joint J along plane(s) P (FIG. 7). Disc 722 has a periphery 726 that defines a thickness t of disc 722. It is envisioned that thickness t may be in a range of 0.5-5.0 mm. Periphery 726 has substantially arcuate configuration and is disposed about the perimeter of disc 722.

Outer surface 724 includes a first lateral surface 728 and a second lateral surface 732. First lateral surface 728 and second lateral surface 732, each have a substantially corrugated configuration. First lateral surface 728 opposes second lateral surface 732 about periphery 726.

Disc 722 is an elastic body formed of an elastic coil having a first end 723 and a second end 725. Disc 722 is coiled such that the circular shape of disc 722 is deformable. The coil of disc 722 is manipulated and deformed from the circular disc shape into a substantially straightened, non-relaxed configuration, as shown in FIG. 18. This configuration of the coil of disc 722 allows for insertion and loading with a dilator/delivery tube 730 and subsequent delivery to sacro-iliac joint J, similar to that described. Disposal of the coil of disc 722 with the dilator/delivery tube 730 maintains the deformed, linear orientation of disc 722. It is contemplated that a plurality of discs 722 may be employed.

In assembly, operation and use, the implant system including sacro-iliac implant 720 is employed with a surgical procedure for treatment of a sacro-iliac joint of a patient and may be employed with other surgical procedures. It is contemplated that sacro-iliac implant 720 may be employed for arthrodesis and/or arthroplasty applications.

Dilator/delivery tube 730 is delivered with sacro-iliac implant 720 and the linear orientation of disc 722 disposed therein, via a protected passageway to joint space JS of sacro-iliac joint J. Disc 722 is unloaded from dilator/delivery tube 730 into joint space JS of sacro-iliac joint J. As sacro-iliac implant 720 enters joint space JS, the coil of disc 722 are no longer subject to the restraint of tube 730 and deformation. The coil returns to the unstressed, relaxed configuration such that disc 722 retains its disc shape, as shown in FIG. 17, due to the compliant configuration of disc 722. Sacro-iliac implant 720 is disposed with sacro-iliac joint J for treating the sacro-iliac joint disorder, similar to that described above. In one embodiment, disc 722 includes a polymeric coil having a smooth, continuous outer surface, which restores its disc shape upon deployment.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A sacro-iliac implant comprising:
at least one circular body defining an outer surface configured to engage at least one articular surface of a sacro-iliac joint along a plane substantially parallel to the articular surface.

2. A sacro-iliac implant according to claim 1, wherein the at least one circular body has a periphery defining a thickness of the body, and the outer surface of the body includes a first side surface and a second side surface.

3. A sacro-iliac implant according to claim 2, wherein at least one of the first side surface and the second side surface defines a concave portion.

4. A sacro-iliac implant according to claim 2, wherein the first side surface and second side surface are each substantially planar.

5. A sacro-iliac implant according to claim 1, wherein the at least one circular body is a disc, the disc including a periphery defining a thickness of the disc, and the outer surface of the body including a first lateral surface and an opposing second lateral surface, at least one of the first lateral surface and the second lateral surface being concave.

6. A sacro-iliac implant according to claim 1, wherein the at least one circular body is a bi-concave disc, the disc including a periphery defining a thickness of the disc, and the outer surface of the body including a first lateral concave surface and an opposing second lateral concave surface.

7. A sacro-iliac implant according to claim 1, wherein the outer surface includes a textured configuration.

8. A sacro-iliac implant according to claim 1, wherein the at least one circular body has a spherical configuration.

9. A sacro-iliac implant according to claim 1, wherein the at least one circular body includes a plurality of spheres.

10. A sacro-iliac implant according to claim 1, wherein the outer surface is configured for movable engagement with the articular surface.

11. A sacro-iliac implant according to claim 1, further comprising a plurality of circular bodies.

12. A sacro-iliac implant according to claim 1, wherein the outer surface is configured to engage opposing articular surfaces.

13. A sacro-iliac implant according to claim 1, wherein the circular body has a diameter dimension greater than a dimension of a joint space defined by opposing articular surfaces of the sacro-iliac joint.

14. An orthopedic implant comprising:
at least one disc defining an outer surface configured to engage at least one articular surface of a sacro-iliac joint along a plane substantially parallel to the articular surface,
the disc including a periphery defining a thickness of the disc, and the outer surface of the disc including a first lateral surface and an opposing second lateral surface.

15. An orthopedic implant according to claim 14, wherein at least one of the first lateral surface and the second lateral surface are concave.

16. An orthopedic implant according to claim 14, wherein the disc is a bi-concave disc such that the outer surface of the disc includes a first lateral concave surface and an opposing second lateral concave surface.

17. An orthopedic implant according to claim 14, wherein the first lateral surface and second lateral surface are each substantially planar.

18. An orthopedic implant according to claim 14, wherein the outer surface is configured for movable engagement with the articular surface.

19. A method for treating a sacro-iliac joint, the method comprising the steps of:
providing a sacro-iliac implant, the implant including at least one circular body defining an outer surface configured to engage at least one articular surface of a sacro-iliac joint along a plane substantially parallel to the articular surface;
defining a trajectory for insertion of the implant within a sacro-iliac joint such that the trajectory is oriented substantially parallel to a plane defined by at least one articular surface of the sacro-iliac joint; and
inserting the implant via the defined trajectory into the sacro-iliac joint such that the outer surface of the body engages the at least one articular surface along the plane defined by the at least one articular surface.

20. A method for treating a sacro-iliac joint according to claim 19, further comprising the step of preparing a cavity in the sacro-iliac joint along the trajectory, the cavity being configured for disposal of the sacro-iliac implant.
